(19) United States
(12) Patent Application Publication
(10) Pub. No.: US 2024/0091531 A1
(43) Pub. Date: Mar. 21, 2024

(54) IMPROVING COGNITIVE FUNCTION

(71) Applicants: THE UNIVERSITY OF BIRMINGHAM, Birmingham, West Midlands (GB); DALHOUSIE UNIVERSITY, Halifax (CA)

(72) Inventors: Sara Assecondi, Birmingham, West Midlands (GB); Kimron Lewis Shapiro, Birmingham, West Midlands (GB); Gail Ann Eskes, Halifax (CA)

(21) Appl. No.: 18/254,153

(22) PCT Filed: Nov. 23, 2021
(86) PCT No.: PCT/GB2021/053019

§ 371 Date: May 23, 2023

(30) Foreign Application Priority Data
Nov. 23, 2020 (GB) 2018397.6

(51) Int. Cl. A61N 1/36; A61B 5/00; A61B 5/374; A61B 5/383

(52) U.S. Cl. CPC A61N 1/36025; A61B 5/374; A61B 5/383; A61B 5/4064; A61N 1/36031

(57) ABSTRACT

A system for improving cognitive function is disclosed. The system comprises a non-invasive brain stimulation device (150); a human computer interface; and a computer (101) configured to control the non-invasive brain stimulation device (150) and the human computer interface. The computer (101) controls the brain stimulation device (150) and the human computer interface to assess a subject's baseline cognitive function by interaction with the subject using the human computer interface. If the subject's baseline cognitive function is impaired, the computer (101) uses the human computer interface to provide the subject with a strategy for improving performance in a cognitive function test and stimulates the subjects brain with the non-invasive brain stimulation device (150) while training the subjects cognitive function.

### FIELD OF THE INVENTION

The present invention relates to a system and a method for improving cognitive function, and more specifically to a method of improving cognitive function comprising non-invasive transcranial stimulation during cognitive training.

### BACKGROUND

Cognitive function can be impaired in a number of ways, for example by acquired brain injury, or by neurodegeneration (which may be a product of the normal aging process, or as a result of a specific disease condition, such as Alzheimer's disease). It is estimated that between 5% and 20% of people over 65 have mild cognitive impairment (MCI), characterized by problems with memory, language, thinking or judgment. MCI is a risk factor in developing Alzheimer's or other forms of dementia.

Acquired Brain Injury is brain damage caused by either a traumatic or a non-traumatic disease or injury (e.g., stroke, brain tumours, ischemia, etc..) that may or may not be related to progressive neurodegeneration. ABI may be associated not only with physical but also with cognitive and behavioural impairments. Depending upon the location of damage, a wide variety of cognitive deficits can result. For example, as brain injury often involves the frontal lobe, affected cognitive domains are likely executive control, working memory, behavioural regulation and problem solving. Other cognitive problems can include memory disruption, attention, concentration, communication and/or visual-spatial problems.

Solutions to improve recovery of cognitive functions that are applicable to all forms of cognitive impairment are of high demand.

Non-invasive brain stimulation has previously been explored as a potential treatment for cognitive impairment due to ABIs. One example is transcranial direct current brain stimulation (tDCS). It has been suggested that tDCS may improve a variety of cognitive functions by exploiting mechanisms of synaptic long-term potentiation and depression. It has been suggested that these mechanisms are particularly efficient when the stimulated area is involved in the cognitive processes under investigation. An interesting application of this principle is in the context of working memory.

Working memory (WM) is a core cognitive function that has been linked to many facets of human cognition, such as attention, memory, language, and general intelligence. WM plays an important role in many aspects of everyday life but is a limited capacity system that declines with age and is compromised by several pathologies, such as epilepsy, schizophrenia, Alzheimer's disease, mild cognitive impairment, and acquired brain injury.

In the prior art, researchers have examined interventions to improve working memory, e.g. by combining task rehearsal and non-invasive brain stimulation. Approaches have included both single- and multi-session designs, with brain stimulation applied concurrently or before a cognitive task and examined in both healthy as well as clinical populations. In spite of its potential, however, outcomes of these studies are inconsistent and highly dependent on methodological parameters. When multisession designs are considered, results are even less consistent across studies.

Although non-invasive brain stimulation remains a promising therapy for treatment of cognitive impairment, there remains considerable room for improvement to the state of the art.

### SUMMARY

According to a first aspect of the invention, there is provided a method of improving cognitive function in a subject comprising:
assessing the subject's baseline cognitive function, and if the subject's baseline cognitive function is impaired:
stimulating the subject's brain by non-invasive brain stimulation while they undertake cognitive function training for improving the impaired cognitive function.

The method may comprise providing the subject with a strategy for improving performance in a cognitive function test prior to cognitive function training. The cognitive function training comprises the cognitive function test for which the subject is provided with a strategy. The provision of a strategy has been found by the inventors to significantly improve the effect resulting from the combination of cognitive function training and non-invasive brain stimulation.

The cognitive function test comprised in the cognitive function training does not necessarily exactly correspond with the impaired cognitive function. It can be envisaged that a working memory test, and cognitive function training comprising a working memory test, may be effective in improving cognitive functions not strictly limited to working memory (e.g. visual problem solving, reading comprehension etc).

The method may further comprise monitoring the subject's cognitive performance during the cognitive function training.

The method may comprise adjusting at least one of: the cognitive function training, strategy for improving performance; and non-invasive brain stimulation in dependence on the result of the monitoring.

Monitoring the subject's cognitive performance may comprise obtaining and processing an EEG.

Monitoring the subjects' cognitive function may comprise monitoring EEG biomarkers that are modulated by brain mechanisms linked to the cognitive function.

Data from the EEG device may be used to position the stimulation cap to provide maximal benefit to the subject

The EEG biomarkers may comprise one or more of: EEG frequency components; power levels within specific frequency ranges; and cross-frequency coupling. The EEG biomarkers may comprise at least one unique pattern of event related potential, or network connectivity. A biomarker comprising network connectivity may comprise a change in correlation (in the frequency or time domain) between sources or sensors of the EEG device.

The EEG may be obtained from a reduced set (fewer than 64, fewer than 32 or fewer than 16) of electrodes, placed according to the international 10-20 electrode system. The EEG may be sampled at a frequency of at least 500 Hz.

The cognitive function may comprise or consist of at least one of: working memory; attention, long term memory and executive control.

The non-invasive transcranial stimulation may comprise electrical transcranial stimulation.

The electrical transcranial stimulation may be applied through two or more electrodes.

The one or more electrodes may comprise silver or silver chloride electrodes. The two or more electrodes may each comprise an area of at least 1 cm 2, or at least 2 cm 2.

The at least two electrodes may be coupled to the subject using a conductive gel.

The at least two electrodes may be placed on positions comprising the F4 and Fp1 positions on the subject (defined with reference to the international 10-20 system). The electrical transcranial stimulation may be applied as a bipolar stimulation via a first and second electrode. The first and second electrode may be applied at the F4 and Fp1 locations.

A current intensity applied during electrical transcranial stimulation may be at least 1 mA, or within the range of 1 mA and 5 mA. An electrical transcranial stimulation period may be at least 10 minutes. An electrical transcranial stimulation period may be between 10 minutes and 30 minutes. A current density during transcranial stimulation at the one or more electrodes may be less than 1 mA/cm².

The electrical transcranial stimulation may comprise at least one of: DC current stimulation; AC current stimulation and random noise stimulation.

The cognitive function training and/or the assessment of the subject's cognitive function may be computer implemented.

The cognitive function training and/or the assessment of the subject's cognitive function may comprise an adaptive computer implemented cognitive function test.

The strategy may comprise an elaborative encoding mnemonic.

The cognitive function test may comprise an nback test for assessment of working memory.

The strategy may comprise stages of:
i) memorizing a working set of n steps of the nback;
ii) comparing the current item with the working set;
iii) updating the working set to discard the oldest member.

The stimulation and training may occur over at least three days (e.g. a 20 minute stimulation and training session on each of three days).

According to a second aspect, there is provided a system for improving cognitive function, comprising:
a non-invasive brain stimulation device;
a human computer interface; and
a computer configured to control the non-invasive brain stimulation device and the human computer interface to:
assess a subject's baseline cognitive function by interaction with the subject using the human computer interface, and if the subject's baseline cognitive function is impaired:
stimulate the subject's brain with the non-invasive brain stimulation device while training the subject's cognitive function.

The computer may be configured to use the human computer interface to provide the subject with a strategy for improving performance in a cognitive function test prior to training the subject's cognitive function.

The non-invasive brain stimulation device may comprise a transcranial electrical brain stimulator.

The transcranial electrical brain simulator may comprise two or more electrodes for applying stimulation. The two or more electrodes may comprise silver or silver chloride electrodes. The two or more electrodes may each comprise an area of at least 1 cm², or at least 2 cm².

The at least two electrodes may be coupled to the subject using a conductive gel.

The transcranial electrical brain simulator may be configured to place the at least two electrodes on positions comprising the F4 and Fp1 positions on the subject (defined with reference to the international 10-20 system). The transcranial electrical brain simulator may be configured to apply electrical transcranial stimulation as a bipolar stimulation via a first and second electrode. The first and second electrode may be configured to be applied at the F4 and Fp1 locations.

The transcranial electrical brain simulator may be configured to apply a current intensity during electrical transcranial stimulation of at least 1 mA, or within the range of 1 mA and 5 mA. An electrical transcranial stimulation period may be at least 10 minutes. An electrical transcranial stimulation period may be between 10 minutes and 30 minutes. A current density during transcranial stimulation at the one or more electrodes may be less than 1 mA/cm².

The transcranial electrical brain stimulator may be configured to apply at least one of: DC current stimulation; AC current stimulation and random noise stimulation.

The computer may be configured to monitor the subject's cognitive performance during the cognitive function training.

The computer may be configured to adjust at least one of: the cognitive function test, strategy for improving performance; and non-invasive brain stimulation in dependence on the result of the monitoring.

The system may further comprise an EEG measurement device, configured to monitor the subject's cognitive performance: i) during assessment of the subject's baseline cognitive performance; and/or ii) while the subject undertakes the cognitive function training.

The system may be configured to use data from the EEG device to instruct positioning of the stimulation cap to provide maximal benefit to the subject.

The EEG measurement device and the non-invasive brain stimulation device may be a unified wearable device.

The EEG measurement device may comprise a reduced set (e.g. fewer than 64, fewer than 32 or fewer than 16) of electrodes, placed according to the international 10-20 electrode system. The EEG measurement device may be configured to sample at a frequency of at least 500 Hz.

The computer may be configured to monitor the subjects' cognitive function using EEG biomarkers that are modulated by brain mechanisms linked to the cognitive function, for example EEG biomarkers comprising one or more of: EEG frequency components; power levels within specific frequency ranges; and cross-frequency coupling.

The cognitive function may comprise or consist of at least one of: working memory; attention, long term memory and executive control.

The system may be configured for operation by the subject in their own home.

The system may be configured to perform the method according to the first aspect, including any of the optional features thereof.

### DETAILED DESCRIPTION

Embodiments of the invention will be described, purely by way of example, with reference to the accompanying drawings, in which:

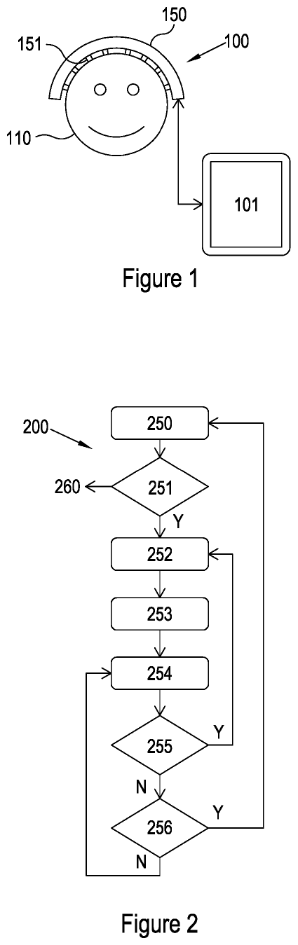

FIG. 1 shows an example embodiment of a system according to an embodiment;

FIG. 2 is a flow diagram of a method according to an example embodiment;

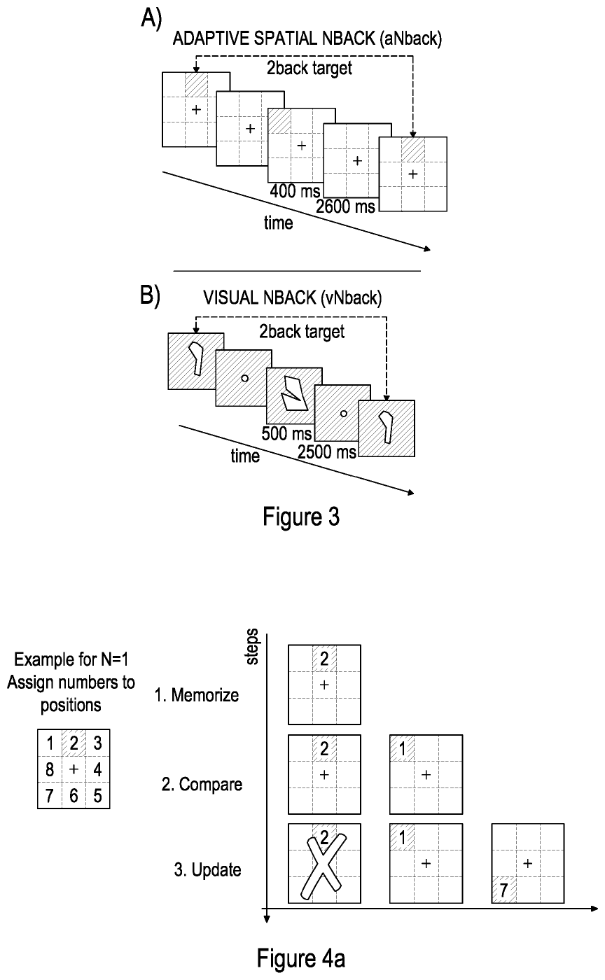

FIG. 3 shows a description of a) an adaptive spatial nback (aNback) test and b) fixed visual nback test (fNback);

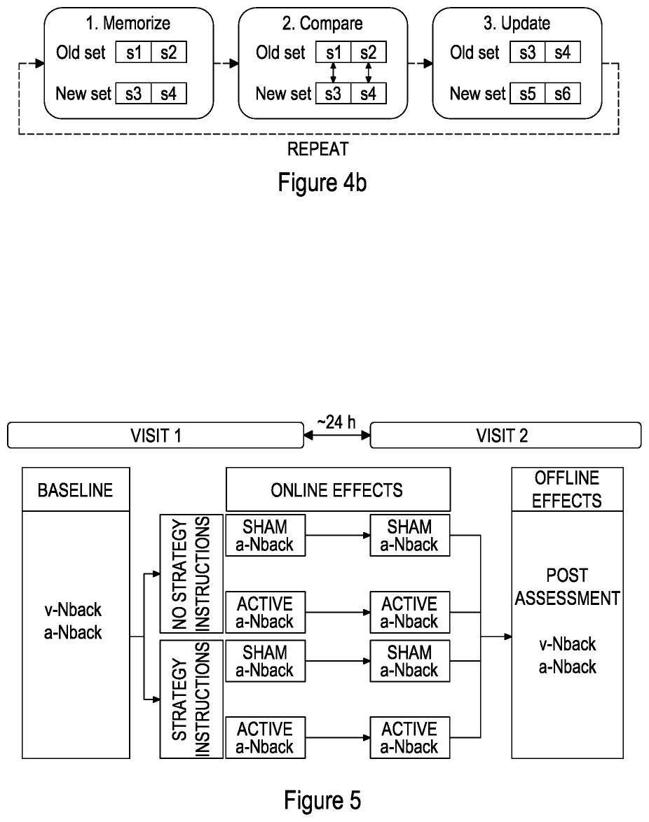

FIGS. 4a and 4b show illustrative strategy instructions for spatial nback working memory testing;

FIG. 5 shows the timeline for an example experiment illustrating a working embodiment;

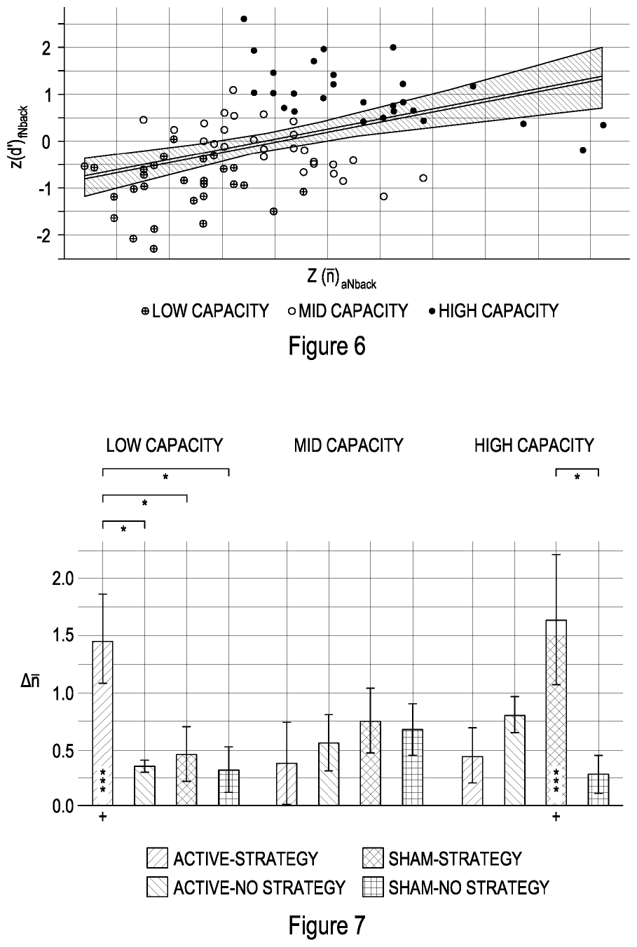

FIG. 6 shows a correlation for baseline cognitive function testing between standardised aNback and fNback testing;

FIG. 7 shows performance change with respect to baseline for each group in the example experiment;

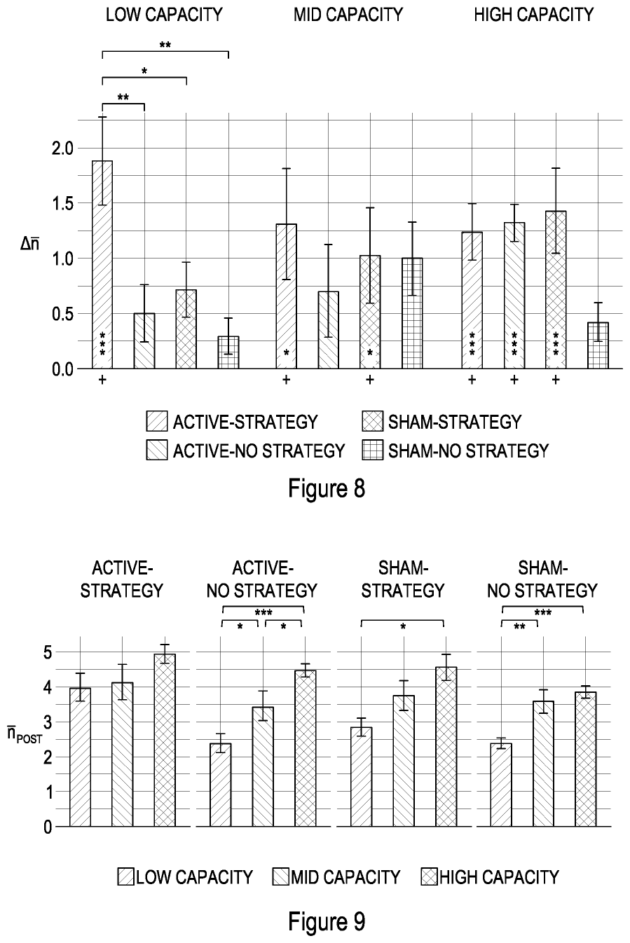

FIG. 8 shows increases in performance across the different groups following the experiment; and

FIG. 9 shows final performance across the different groups following the experiment.

Referring to FIG. 1, an example system 100 is shown for improving cognitive function, comprising a computer 101 and non-invasive brain stimulation device 150.

The computer 101 in the example embodiment is a tablet, provided with a human computer interface in the form of a touch screen, microphone and speaker. In other embodiments the computer may be remote from the user (e.g. a cloud based server instance, or a remote desktop computer), and the human computer interface may comprise peripheral devices such as a screen, keyboard, mouse, speakers, microphone etc. Any suitable computing device such as a laptop, mobile phone etc can be used as the computer 101.

The non-invasive brain stimulation (NIBS) device 150 is configured to stimulate the subject's brain non-invasively. In the example embodiment the non-invasive brain stimulation device 150 comprises a transcranial electrical stimulation (tDCS) device, which comprises electrodes 151. The electrodes 151 are configured to make contact with the user's head and transmit electrical currents to/from the user's scalp. The NIBS device 150 may comprise a power source and a control electronics that are configured to cause specific currents to be imparted to the user (e.g. specific waveforms and/or current flows, directed to specific regions of the brain). The NIBS device 150 is configured to communicate with the computer 101, which may control the NIBS device 150 in accordance with the overall method, as explained in more detail below. In some embodiments the NIBS device 150 may be configured to communicate with the computer 101 via a wired connection (e.g. by a USB interface). In some embodiments the NIBS device 150 may be configured to communicate with the computer 101 wirelessly so that it can be controlled remotely. Examples of suitable wireless communication include wifi, Bluetooth, and Zigbee. The NIBS device 150 may comprise a plurality of electrodes 151, and may be configured to enable safe stimulation between electrodes that are selected in response to control signals provided to the NIBS device 150 (e.g. by wireless communication from the computer 101). The type of stimulation (e.g. AC, DC, noise), current flow and electrode pairs may be selectable.

The NIBS device 150 may be configured for home use, so that it cannot be used to impart an unsafe stimulation to a subject's brain. For example, current flow may be limited to a predetermined safe threshold level, and authentication may be required in order to limit use to a specific subject.

In some embodiments, the system 100 may comprise an EEG device, for monitoring the brain activity of the subject. In the depicted example, the EEG device is integrated with the NIBS device 150, so that the same wearable device can provide both stimulation and EEG monitoring. The EEG information may be interpreted by the computer 101 in order to inform, define and adjust the approach employed by the system 100 to enhance cognitive function in the subject. Similarly to the NIBS device, the EEG device may be connected to the computer 101 with a wired or wireless communication channel. The EEG device may be remotely controllable and have the ability to upload data to the computer 101 and/or to a remote server (suitably secured). Data from the EEG device may be used to position the stimulation cap to provide maximal benefit to the subject.

The system 100 may be configured for self-administered use (e.g. in a home setting). In other embodiments, the system 100 may be configured for use by a medically qualified person (e.g. nurse practitioner/doctor etc.), or under the supervision of a medically qualified person.

FIG. 2 illustrates an outline of a method 200 for improving cognitive function, which the system 100 may be configured to implement.

The method 200 starts with a step 250 of cognitive assessment, in which the cognitive performance of the subject is assessed. The assessment may comprise more than one methodology. Examples of suitable cognitive function tests include: the Montreal Cognitive Assessment test¹, Mini-mental State Exam², Mini-Cog³, nBack working memory testing, the MATRICS⁴ Consensus Cognitive Battery, IntegNeuro⁴ and DalCAB⁵.

The step 250 of cognitive assessment may be computer implemented, with the subject answering questions or completing tasks that are set by the computer via a human-computer interface (e.g. tablet interaction via a touchscreen). For tasks that involve movement, a motion capture camera may be used to capture the subject's task performance. In addition to cognitive assessment by obtaining the subject's response to

1 Nasreddine, Ziad S., et al. “The Montreal Cognitive Assessment, MoCA: a brief screening tool for mild cognitive impairment.” Journal of the American Geriatrics Society 53.4 (2005): 695-699.
2 Folstein, Marshal F., Lee N. Robins, and John E. Heizer. “The mini-mental state examination.” Archives of general psychiatry 40.7 (1983): 812-812.
3 Berson, Soo, et al. “The mini-cog: a cognitive ‘vital signs’ measure for dementia screening in multi-lingual elderly.” International journal of geriatric psychiatry 15.11 (2000): 1021-1027.
4 Silverstein, Steven M., et al. “A comparative study of the MATRICS and IntegNeuro cognitive assessment batteries.” Journal of Clinical and Experimental Neuropsychology 32.9 (2010): 937-952.
5 Jones, Stephanie AH, et al. “Measuring the performance of attention networks with the Dalhousie Computerized Attention Battery (DalCAB): Methodology and reliability in healthy adults.” Frontiers in psychology 7 (2016): 823. task and question based cognitive testing (which may include their reaction times and accuracy), physiological parameters of the subject may be monitored, either during the cognitive testing or separately. The physiological parameters may include EEG measurements (and biomarkers derived from EEG measurements), which can be used as part of a baseline cognitive assessment of the subject.

The result of the cognitive assessment step 250 is a report of baseline cognitive function of the subject, which will record the subject's cognitive functioning in at least one cognitive domain. For example, each cognitive domain may be assigned a score. In some embodiments a composite score for the subject's overall cognitive function may be determined, for example from a weighted sum of the cognitive function score in each domain. The cognitive assessment step 250 may also identify one or more biomarkers that can be used to monitor cognitive function in each domain of interest. Such biomarkers may include EEG features. Suitable EEG features include:
i) oscillations: frequency, power in a particular frequency range, frequency cross-coupling
ii) short and long range connectivity, which may be quantified by cross-frequency coupling within particular frequency ranges, or between frequencies.

EEG monitoring may be informed by the baseline test, so that electrode placement during training can be targeted to enable monitoring of biomarkers that are specific to a particular cognitive impairment. The appropriate electrode positioning may be based on known brain areas that serve specific cognitive functions that have been identified as impaired by the baseline assessment in step 250.

The tests may assess functional ability in at least some of the following domains:
i. Attention: the ability to concentrate, select and allocate processing resources to the relevant stimulus while ignoring other goal-irrelevant items.
ii. Short-term (working) memory: the ability to retain (short-term memory) and manipulate (working memory) limited information for a limited time.
iii. Long-term memory: the ability to recall information over a longer term, such as days, weeks or months.
iv. Executive control: the ability to plan, organise and monitor appropriate behaviours to achieve a set goal.

An assessment version of the cognitive tests may be used to assess baseline performance, and a corresponding adaptive algorithmic version of each test may be used to train each domain of cognitive ability.

At step 251, a determination is made as to whether there are any cognitive domains in which the subject has a cognitive impairment. A determination of impairment may be based on a statistical definition of unimpaired baseline performance. For example, impaired function may be associated with a lower percentile performance in a particular domain (between 0 and 33^{rd} percentile, or 0-25^{th} percentile, or 0-10^{th} percentile). Impaired function in the present application may be sub-clinical. The determination step identifies in which domains of cognitive function (if any) the subject has an impairment (or low capacity). It is these domains that can be subsequently improved. Step 251 therefore comprises comparing a subject's baseline cognitive function in at least one domain with a healthy or normal range to determine whether there is impairment (which may be sub-clinical). In addition, step 251 may comprise testing for physiological biomarkers indicative of cognitive impairment (e.g. obtained via EEG).

At step 252, a training regime is determined with the aim of improving cognitive function in the domains that are identified as impaired (from step 251). The computer 101 is configured to determine type of exercise, frequency, initial difficulty, NIBS configuration and any physiological monitoring (e.g. by EEG).

For example step 251 may identify an impairment in working memory. At step 252 the computer may select one or more working memory training exercises (which are directed to improving cognitive function in the impaired domain) that are to be performed at regular intervals (e.g. daily). The computer 101 may determine how to configure the NIBS device 150 to stimulate a region of the subject's brain that is associated with the impaired domain while the subject is performing the brain training exercises.

The number of training sessions and their duration may be selected in dependence on the degree of impairment. For example, a subject with working memory in the 10t h percentile may have more scheduled training sessions than a subject with working memory in the 20t h percentile. Similarly, the amount of improvement during training may affect how many training session are subsequently needed.

The stimulation parameters may be selected as part of the training regime and appropriate stimulation and electrode location parameters may be determined. Two electrodes may be used: an active electrode with a positive current and a return electrode with a negative current and the location of these electrodes selected based on the cognitive domains that are identified as impaired. The current intensity may be selected from standard values that depend on the type of stimulation (DC, AC, noise etc.). The current intensity may be modified based on monitored physiological parameters, such as EEG biomarkers.

The form of stimulation chosen may be based on the cognitive domain that is impaired and the best approach for achieving modification of that impairment, e.g., tDCS for modifying plasticity, tACS for entraining specific frequencies and stimulating one or multiple brain areas.

At step 253 the computer 101 may provide the subject with a strategy for improving the performance of the subject in each impaired domain. The inventors have found that the provision of a strategy to the subject is important in achieving significant improvement in cognitive function. The subject is prompted (e.g. by the computer) to think of the importance of devising a task strategy and how to maximise the utility of any task strategy that is provided by the computer 101. The strategy may provide a visual or auditory mnemonic. An example of a strategy for improving performance in the domain of working memory is an elaborative encoding mnemonic, in which information to be remembered is related to previously existing memories or knowledge. Examples include: encoding spatial information as an alphanumeric sequence, method of loci, mnemonic peg system etc.

The selected strategy may be tailored to the specific subject, based on their individual strengths and weaknesses and/or on prior empirically determined knowledge of the efficacy of different training strategies. The identification of a strategy may boost the efficacy of brain stimulation by targeting the relevant brain circuits active during a chosen training task. The identification of cognitive deficiency using EEG biomarkers may further inform the position and protocol of the brain stimulation device, as well as monitoring treatment efficacy, including session length and number.

At step 254 the subject is subjected to a cognitive test (or brain training exercise) in accordance with the training regime determined in step 252. At the same time as administering the cognitive function test, the computer 101 controls the NIBS device 150 to stimulate the subject's brain in accordance with the training regime from step 252. The computer 101 may monitor the subject's physiological parameters (e.g. by EEG and monitoring EEG biomarkers) during testing. The results of the testing will provide data as to the cognitive function of the subject, which will indicate whether there is a change in performance in any of the domains previously identified as impaired.

Individuals may be able to commence cognitive training with an automatically identified brain stimulation configuration depending on their individual needs. Electrophysiological biomarkers may be monitored throughout the training. Based on biomarkers and behavioural performance, an algorithm (implemented by the computer 101) may adjust the testing/training to suit the subject's individual needs and to maximise training efficacy while reducing training time. For example, if the subject is improving in attention but not in memory, the system may offer more training in memory while reducing the attention component of training. By comparing with norms (e.g. from a young and healthy population), the system may determine when the subject requires a break from training and/or identify when it is appropriate to change the interval between training sessions.

At step 255, the change in performance in each targeted cognitive domain may be assessed. For example a change in performance may be compared with a predetermined expected performance change, calculated based on the information that is known about the subject (e.g. based on a statistical model of typical improvement in similar users). In another example, performance gains in each domain may be compared to see if there is a particular domain in which improvement is slower or not occurring. For each domain, a determination may be made as to whether adjustment is needed to either the testing/training regime or the stimulation. If a determination is made that adjustment is required, the method returns to step 252, where the training regime is adjusted.

Parameters of the cognitive test, strategy and/or non-invasive brain stimulation may be adjusted during testing (between testing sessions and/or during testing sessions). For example, if the monitoring step 255 indicates that cognitive function in a specific domain is improving, the difficulty of a test/training may be increased. In another example, if no improvement in a targeted domain is occurring, an alternative strategy may be provided to the subject and/or the brain stimulation may be adjusted. For example, in the case of tDCS, the amount of current may be adjusted, and/or the placement of active electrodes (used for stimulation) varied.

At step 256, the performance (and optionally, biomarkers) of the subject may be assessed to see if the objectives of the method are met. In some embodiments, the objective may be a return to cognitive function that is not impaired (e.g. at 33^{rd} percentile or greater, or above whatever definition is used for “impaired”). In other embodiments, a convergence in cognitive performance over time to a new baseline level may indicate that there is no further benefit to continuing. Biomarkers being within normal parameters may also indicate that the impaired cognitive functions have improved.

If the objectives of the method are met, the cognitive training/testing can be suspended, and a monitoring regime can be entered, in which cognitive performance is monitored relatively infrequently (e.g. once a month) to see if further stimulation and testing/training would be of benefit. If the objectives of the method are not met, training/testing and stimulation can be continued.

### Example experiment

An example of an experiment to show the efficacy of an embodiment of the invention will be described below.

Participants

Ninety-two (65 female) right-handed participants (Mean age=20.6±3.8, range 18 to 39) were recruited. Participants who did not fulfil safety inclusion criteria for brain stimulation, had a history of depression, or had received brain stimulation or cognitive training in the previous 6 months were not eligible for the study. Eight participants dropped out after the first day, resulting in a total of 84 participants. The research procedures were subject to ethical approval in accordance with the Declaration of Helsinki. All participants gave their informed consent before starting the study.

Method

A between-subject design for the experiment was used, with STIMULATION (2 levels), STRATEGY (2 levels) and CAPACITY (3 levels) as independent factors. Details of each analysis are provided in the results section. Participants were assigned randomly to two groups, receiving either SHAM or ACTIVE stimulation. Within those groups, participants were further randomly divided into two groups, one receiving strategy instructions (in accordance with an embodiment), and one that did not receive strategy instructions (not in accordance with an embodiment).

The targeted domain in the experiment was working memory, with an nback test used to both assess baseline performance and used for cognitive function training.

An adaptive spatial n-back paradigm was used in 2 training sessions, with concurrent tDCS of the right dorsolateral prefrontal cortex (DLPFC). We evaluated the impact of strategy and stimulation by comparing task performance from pre- to a post-session (e.g., without tDCS) on the same adaptive spatial n-back and on a fixed-load visual n-back, to tease apart the effects of brain stimulation and strategy development.

Transcranial Direct Current Stimulation

Transcranial direct current stimulation (tDCS) was administered using an 8-channel device (Starstim, Neuroelectrics®). Participants received stimulation during two practice sessions over two consecutive days via two circular Ag/AgCl electrodes (NG Pistim, Neuroelectrics) of 1 cm radius (3.14 cm 2 area). Electrode impedance was kept below 10 kOhm by using a conductive gel (SignaGel, ParkerLabs) between electrodes and scalp. The anode was placed over the right dorsolateral prefrontal cortex (rDLPFC, F4) with the cathode over the contralateral supraorbital site (Fp1), according to the international 10-20 system. In the ACTIVE group the current was ramped up to 2 mA (current density=0.64 mA/cm 2) in the first 30 seconds and maintained for 20 minutes before ramping down to 0 mA in the last 30 seconds (total ACTIVE time 21 minutes). In the SHAM group current was ramped up to 2 mA in the first 30 seconds then immediately ramped down to 0 mA in the next 30 seconds, where it was maintained for 20 minutes followed by another cycle of ramping up and down (total SHAM time 21 minutes). Participants were randomly assigned to either the ACTIVE or the SHAM tDCS condition and both blinding and side effects were monitored via a feedback questionnaire

Adaptive Spatial Nback Task (aNback)

In the adaptive N-back working memory task (FIG. 3, panel A)) participants were told to monitor the position of a sequence of blue squares appearing randomly in one of the 8 positions defined by a 3×3 grid on the screen (centre position excluded), and to report whether the current location matched a previous one ‘n’ trials ago. Each square was presented for 0.4 seconds, followed by an empty grid for 2.6 seconds (total trial length 3 seconds). Participants were asked to be as accurate and as fast as possible, responding to match (target) trials by pressing the key “A”, whereas non-match (non-target) trials did not require a response. Participants received feedback on their accuracy every time a response was given. Starting from a difficulty of n=2 (indicating ‘n’ items to remember), as participants' performance score (True Positives/(True Positives+False Positive+False Negatives)) was at or above 70% in a block, difficulty would increase by one, or decrease by one if it was below 50%. Each training session consisted of 15 blocks or sequences of 20+n trials each. Each block of trials included 12.5% matches and 12.5% interference (e.g., 12.5 of non-match trials presented the target item at position n-1 or n+1 instead of n). At the end of each block participants received feedback on performance and the task was appropriately adapted for the subsequent block.

Fixed-Load Visual Nback Task (INback)

Participants' visual working memory was assessed on a nonadaptive visual NBACK task with random shapes (see FIG. 3, Error! Reference source not found.Panel B). Stimuli consisted of 10 random shapes presented sequentially in the centre of the screen for a total of 0.5 seconds, followed by a fixation cross for 2.5 seconds (total trial length 3 seconds). Participants were asked to be as accurate and as fast as possible while searching for shapes matching with ‘n’ items before, responding by pressing “A” for matches and “L” for no matches. Participants completed 6 blocks, or sequences, for n=2 and 6 blocks for n=3, for a total of 120 trials for each n. Each sequence included 30% matches and 10% interference (as defined above).

Strategy Instructions and Questionnaires

Before starting the first session with concurrent tDCS (ACTIVE or SHAM) and the adaptive nback training task, half of the participants were provided with clear instructions on how to undertake the working memory training. The strategy follows is depicted in FIG. 4. Participants were first asked to associate a number to each of the 8 positions on the screen and were reminded that the central position was unavailable. Then they were briefed on the use of a strategy based on “memorize”, ‘compare’ and ‘update’.

FIG. 4a illustrates this scheme, in which numbers are assigned to the different spatial positions. The sequence of spatial positions can be encoded as a sequence of numbers (an example of an elaborative encoding mnemonic), which can be memorised. A new spatial position appearing on the screen can be encoded as a new number, which can be compared with the memorised sequence to determine whether it matches with an item ‘n’ positions ago (i.e. nBack). The memorised sequence is subsequently updated by discarding the least recent position (encoded as a number) and adding the most recent position (encoded as a number). Participants in the no-strategy group were introduced to the task and instructed to “do their best trying to get to the highest possible n level”.

FIG. 4b further illustrates a strategy for nback training, comprising the “memorize”, “compare” and “update” steps. Subjects may be instructed to create a memory array with the first ‘n’ items in the stream, then to create a second array with the next ‘n’ items. At this point they can compare ‘new’ items with ‘old’ ones and respond. Finally, they discard the ‘old’ array, not necessary, and create a new one. The process was repeated until the end of the stream.

Mood and motivation were monitored by administering the Positive Affect/Negative Affect Schedule (PANAS) at the beginning of the baseline session and at the end of the post-assessment, in addition to 5 additional questions on a Likert scale (1 to 5, on alertness, motivation, sadness and expectation on both the working memory performance and the effect of tDCS) to be answered based on one's subjective ‘feeling’ before each administration of the adaptive Nback task.

Procedure

The timeline of the experiment is shown in FIG. 5. Participants completed the study over two visits on two consecutive days. During the first visit, all participants underwent the baseline assessment, in the following order: PANAS, one session of the fNback, and one session of the aNback. The STRATEGY group received detailed instructions on a specific strategy proven to be task beneficial, while those in the NoSTRATEGY group were not instructed to develop a strategy. Both groups were encouraged to achieve as high an ‘n’ as possible. Following the administration of strategy instructions (according to the respective group), participants were prepped for brain stimulation and undertook another session of the aNback with either SHAM or ACTIVE tDCS, which completed the first visit. On the second visit the following day, participants first completed a session of the aNback with tDCS (ACTIVE or SHAM), followed by the post-assessment including in the following order: one session of the aNback (without tDCS), one session of the fNback, and the PANAS. The two tDCS sessions provided data on the online effects of combining tDCS with strategy instructions, whereas offline effects were quantified during the post-assessment. Before each session of the aNback task, participants completed a motivation and expectation questionnaire, and a feedback form on the side effects of tDCS after each brain stimulation session (both ACTIVE and SHAM). At the end of the second visit they also completed a feedback form on the strategy used during both tasks and answered some questions about the blinding.

Results—Initial Baseline Data

A 1-way independent ANOVA showed that the four groups did not differ in age, gender distribution, years of education, motivation, mood, attitude or baseline performance (all ps>0.05, see Supplementary Material). A chi-square test of independence showed no significant association between actual and perceived stimulation, indicating that subjects were blind to the stimulation group (X²_{(1, N=84)}=0.86289, p=0.3529).

Working Memory Capacity Scores

A relationship was found between participant's baseline performance and the outcome of the intervention. Participants were grouped into high-, mid- and low-capacity, based on their memory capacity score when entering the study. For each participant, a composite capacity score was calculated as the mean of their aNback and fNabck standardized scores at baseline.

Standardized scores were calculated across the entire sample, as follows: z-transform scores against the mean ‘n’ for the aNback, and z-transform of d-prime for the fNback, averaged for n=2 and n=3. FIG. 6 shows the correlation between these scores, and shows that scores for the two tasks were significantly related but shared only a small amount of variance (r p=0.43, p<0.001).

Composite capacity scores were split into three groups: low capacity between 0 and 33^{rd} percentile, mid capacity 34^{th} and 66^{th} and high capacity between 67th and 100th percentile. No differences in capacity scores were found between the four groups, when the percentiles are aggregated (STIMULATION×STRATEGY, p>0.1). There was no significant association between the groups and the capacity membership (X²(6)=2.788, p=0.835). Thus, there were no factors compromising the results of the independent variables under investigation.

Online Effects of the Intervention

To evaluate the online effect of tDCS and strategy instructions, changes in performance during training across the two tDCS sessions in the aNback task were quantified as the average difference between the mean ‘n’ within a session (excluding the first block) and the mean ‘n’ at baseline (Δη=η−η_{baseline}). The hypothesis to be tested was that combination of tDCS and strategy instructions would be particularly beneficial in the low capacity group. To test this hypothesis, a 4-way mixed ANOVA was conducted, with 3 between-subject factors (STIMULATION: ACTIVE, CONTROL x STRATEGY: STRATEGY, NoSTRATEGY x CAPACITY: LOW, MID, HIGH) and one within-subject factor (TIME: change at DAY 1, DAY 2). Main effects of TIME (F_{(1,72)}=35.207, p<0.001, ηₚ²=0.328) and STRATEGY (F_{(1,72)}=4.813, p=0.031, ηₚ²=0.063) were found, and significant interactions of TIMEX STRATEGY (F_{(1,72)}=5.495, p=0.022, ηₚ²=0.071) and STIMULATION x STRATEGY x CAPACITY (F_{(2,72)}=5.817, p=0.005, ηₚ²=0.139). Multiple comparisons were conducted between groups (t-test, Holm-corrected) within each CAPACITY level (LOW, MID, HIGH), after collapsing across TIME.

The low-capacity, ACTIVE-STRATEGY group achieved performance improvements significantly larger than zero (t₍₂₀₎=6.105, p<0.001) and larger than the other 3 groups (ACTIVE-NoSTRATEGY: t₍₄₀₎=3.482, p_{H}=0.012, d_{Cohen}=1.605; SHAM-STRATEGY: t₍₄₀₎=2.858, p_{H}=0.035, d_{Cohen}=1.165; SHAM-NoSTRATEGY: t (40)=3.243, p_{H}=0.017, d_{Cohen} −1.364). No significant differences between groups were found in individuals with mid-capacity (ps>0.1), whereas the high-capacity SHAM-STRATEGY group's improvement was significantly greater than zero (t₍₂₀₎=5.016, p<0.001) and larger than the SHAM-NoSTRATEGY group (t₍₄₀₎=3.216, p_{H}=0.021, d_{Cohen}=1.416).

FIG. 7 shows performance changes (Δη), collapsed across the two online sessions (TIME), with respect to baseline, split by CAPACITY. Means with standard errors are reported for each group and capacity. P-values are marked as follows: * p<0.05, ** p<0.01, *** p<0.001. Changes significantly larger than zero are marked with +.

A 1-way ANOVA to compare differences in the working memory capacity groups in each of the four conditions (ACTIVE vs SHAM, STRATEGY vs NoSTRATEGY), using the mean ‘n’ of the last tDCS session (n, without baseline subtraction) as the dependent variable, revealed that the low and high capacity groups showed equivalent performance (i.e., not significantly different from each other) but only in the ACTIVE-STRATEGY group (ps>0.5).

Offline Effects of the Intervention

To examine the overall effects of the intervention on working memory (offline effects) after two sessions of brain stimulation, the changes in performance in the aNback task (Δη), in the post-assessment were analysed compared to the baseline. Again, the hypothesis to be tested is that the combination of tDCS and strategy instructions is particularly beneficial in the low-capacity group. A 3-way independent ANOVA (STIMULATION: ACTIVE, CONTROL x STRATEGY: STRATEGY, NoSTRATEGY x CAPACITY: LOW, MID, HIGH) revealed a significant main effect of STRATEGY (F_{(1,72)}=8.395, p=0.005, ηₚ²=0.104) with the STRATEGY group achieving larger improvement than the NO STRATEGY group (Dc=0.63), and a main effect of STIMULATION trending towards significance (F_{(1,72)}=3.191, p=0.078, ηₚ²=0.042), with the ACTIVE group achieving larger performance improvements than the SHAM group (Dc=0.374). A 3-way interaction was found of STIMULATION x STRATEGY x CAPACITY trending towards significance (F_{(2,72)}=2.711, p=0.073, ηₚ²=0.070).

As the hypothesis is specific to individuals with low-capacity and the effect size of the 3-way interaction is medium to large, the 3-way interaction was further investigated with planned multiple comparisons between groups (t-test, Holm-corrected), within each CAPACITY level (LOW, MID, HIGH). In individuals with low-capacity, the ACTIVE-STRATEGY group achieved offline performance improvements significantly larger than zero (t₍₂₀₎=6.408, p<0.001) and larger than the other 3 groups (ACTIVE-NoSTRATEGY: t (40)=3.566, p_{H}=0.008, d_{Cohen}=1.522; SHAM-STRATEGY: t (40)=2.728, p_{H}=0.047, d_{Cohen}=1.314; SHAM-NO STRATEGY: t (40)=3.705, p_{H}=0.007, d_{Cohen}=1.916).

No significant differences from zero or between groups were found within individuals with mid-capacity (ps>0.1). In individuals with high-capacity, offline performance changes significantly larger than zero were found in the ACTIVE-STRATEGY (p<0.001, t (20)=5.279), the ACTIVE-NoSTRATEGY (p<0.001, t (20)=4.868) and the SHAM-STRATEGY (p<0.001, t (20)=5.263), while only the SHAM-STRATEGY group achieved improvements in performance significantly larger than the SHAM-NoSTRATEGY group (t (40)=2.876, p_{H}=0.049, d_{Cohen}=1.417).

FIG. 8 shows increase in mean ‘n’ (Δη) on the aNBack at the offline POST-ASSESSMENT with respect to BASELINE. Means with standard errors are reported for each group and capacity performance changes at session 4 with respect to baseline, split by CAPACITY. Means with standard errors are reported for each group and capacity. P-values are marked as follows: * p<0.05, ** p<0.01, *** p<0.001. Changes significantly larger than zero are marked with +.

To investigate the overall ‘n’ achieved after all the sessions, a 1-way ANOVA (CAPACITY) was performed in each group (ACTIVE vs SHAM, STRATEGY vs NoSTRATEGY), using the mean ‘n’ of the post-assessment run (n, without baseline subtraction) as the dependent variable. Only in the ACTIVE-STRATEGY group did we difference found in n between individuals with low, mid and high capacity (ps>0.1). Every other group showed a significantly lower n in individuals with low capacity than in individuals with high capacity (ps<0.05). Thus, only in the ACTIVE-STRATEGY group did individuals with low capacity achieve performance equivalent to (i.e., not significantly different from) individuals with high capacity.

FIG. 9 illustrates this, showing a comparison of the mean ‘n’ (fi) reached in the POST-ASSESSMENT on the aNBack. P-values are marked as follows: * p<0.05, ** p<0.01, *** p<0.001.

Retrospective strategy analysis and other questionnaires No modulation of mood, alertness, sadness or expectations was found, driven either by brain stimulation, strategy instructions or their interaction (see supplemental material for a full description). To validate the strategy manipulation, at the end of day 2, participants were asked to report the strategy used in each run. After strategy instructions, only one participant in the ACTIVE-STRATEGY group failed to use the strategy after the first session, with two participants in the CONTROL-STRATEGY group reporting a failure to adopt the strategy. No difference was found in the strategy used at baseline between the four groups (X²₍₃₀₎=29.634, p=0.484) or between capacities (X²₍₂₀₎=21.870, p=0.348).

### DISCUSSION

The experiment demonstrated the hypothesis: that embodiments can improve cognitive function. More specifically, it is shown that the provision of a strategy increases the effectiveness of transcranial direct current stimulation on a spatial WM task. The combination of strategy and stimulation is particularly beneficial for individuals with initially low working memory capacity. It is believed (and is plausible) that a similar mechanism will provide a synergistic effect between the provision of a strategy and training of other cognitive domains concurrent with brain stimulation (including other stimulation modalities).

Individual differences in age and education influence WM baseline abilities, which in turn impact an individual's responsiveness to cognitive tasks and the ability to benefit from stimulation induced plasticity. The aptitude-by-treatment interaction theory states that the outcome of treatment is modulated by individual factors. Relevant to the current study, one of these factors is the ability to derive an efficient strategy.

Consistent with the above factors, the combination of strategy instructions and tDCS improved WM over and above either strategy or stimulation alone but only in individuals with low baseline capacity. Individuals with high capacity likely have the cognitive resources to devise a strategy and adapt it to increasing task difficulty, whereas low-capacity individuals require additional resources to use the strategy provided, which was facilitated by tDCS-induced plasticity.

Importantly, individuals with low WM capacity maintained the advantage conferred by combined strategy instructions and brain stimulation after the stimulation ended (post-assessment offline session, see FIG. 8). Perhaps even more importantly, participants in the ACTIVE-STRATEGY low-capacity group achieved performance comparable to participants with high-capacity (see FIG. 9).

Individuals undertaking a cognitive task are likely to devise a strategy they deem efficient. However, the time required to develop a strategy is highly variable and cognitively demanding, potentially nulling the positive effects of brain plasticity.

Devising a strategy requires resources that are taken away from the task, as participants use some of the inter-stimulus time to make an effective choice of strategy. Thus, when a strategy is provided, participants can construct a better representation of the visual stimuli, supported by the right DLPFC and further enhanced by stimulation over that region. Second, the right DLPFC has connections with other brain regions, therefore stimulation may augment brain areas subserving verbal working memory, such as the left DLPFC.

The present study reveals that initial skill set is linked to task outcome and sets limits on the effectiveness of brain stimulation. Importantly, the present disclosure has implications for training regimens in a general sense, e.g., encouraging designing interventions predicated on baseline skill set, or interventions focusing on strategy development for specific attentional skills in addition to than task repetition.

The example experiment focused on young participants. Evidence shows that older adults use different cognitive resources in a working memory task with respect to young adults, with older adults making a more extensive use of attention, verbal memory and updating than their younger counterparts. It would therefore not be surprising that older adults may benefit even more from strategy instructions than young participants, especially those whose baseline performance is impaired due to normal or abnormal ageing.

The example embodiments described herein are not intended to limit the scope of the invention, which is based on the appended claims. The skilled person will understand that variations of the example embodiments are possible, within the scope of the appended claims.

1. A system for improving cognitive function, comprising:
a non-invasive brain stimulation device;
a human computer interface; and
a computer configured to control the non-invasive brain stimulation device and the human computer interface to:
assess a subject's baseline cognitive function by interaction with the subject using the human computer interface, and if the subject's baseline cognitive function is impaired:
use the human computer interface to provide the subject with a strategy for improving performance in a cognitive function test; and
stimulate the subject's brain with the non-invasive brain stimulation device while training the subject's cognitive function.

2. The system of claim 1, wherein the non-invasive brain stimulation device comprises a transcranial electrical brain stimulator.

3. The system of claim 2, wherein the transcranial electrical brain stimulator is configured to apply at least one of: DC current stimulation; AC current stimulation and random noise stimulation.

4. The system of any of claims 1 to 3, wherein the computer is configured to monitor the subject's cognitive performance during the cognitive function training.

5. The system of any of claims 1 to 4, wherein the computer is configured to adjust at least one of: the cognitive function test, strategy for improving performance; and
non-invasive brain stimulation in dependence on the result of the monitoring.

6. The system of any of claims 1 to 5, further comprising an EEG measurement device, configured to monitor the subject's cognitive performance: i) during assessment of the subject's baseline cognitive performance; and/or ii) while the subject undertakes the cognitive function training.

7. The system of claim 6, wherein the EEG measurement device and the non-invasive brain stimulation device are a unified wearable device.

8. The system of claim 6 or 7, wherein the computer is configured to monitor the subjects' cognitive function using EEG biomarkers that are modulated by brain mechanisms linked to the cognitive function, for example EEG biomarkers comprising one or more of: EEG frequency components; power levels within specific frequency ranges; and cross-frequency coupling.

9. The system of any of claims 1 to 8, wherein the cognitive function comprises or consists of at least one of: working memory; attention, long term memory and executive control.

10. The system of any of claims 1 to 9, wherein the system is configured for operation by the subject in their own home.

11. A method of improving cognitive function in a subject comprising:
assessing the subject's baseline cognitive function, and if the subject's baseline cognitive function is impaired:
providing the subject with a strategy for improving performance in a cognitive function test; and
stimulating the subject's brain by non-invasive brain stimulation while they undertake cognitive function training.

12. The method of claim 1, further comprising monitoring the subject's cognitive performance during the cognitive function training.

13. The method of claim 12, further comprising adjusting at least one of: the cognitive function training, strategy for improving performance; and non-invasive brain stimulation in dependence on the result of the monitoring.

14. The method of claim 12 or 13, wherein monitoring the subject's cognitive performance comprises obtaining and processing an EEG.

15. The method of claim 14, wherein monitoring the subjects' cognitive function monitoring EEG biomarkers that are modulated by brain mechanisms linked to the cognitive function.

16. The method of claim 15, wherein the EEG biomarkers comprise one or more of: EEG frequency components; power levels within specific frequency ranges; and cross-frequency coupling.

17. The method of any of claims 11 to 16, wherein the cognitive function comprises or consists of at least one of: working memory; attention, long term memory and executive control.

18. The method of any of claims 1 to 17, wherein the non-invasive transcranial stimulation comprises electrical transcranial stimulation.

19. The method of claim 18, wherein the electrical transcranial stimulation comprises at least one of: DC current stimulation; AC current stimulation and random noise stimulation.

20. The method of any of claims 11 to 19, wherein the cognitive function training and/or the assessment of the subject's cognitive function is computer implemented.

21. The method of claim 20, wherein the cognitive function training and/or the assessment of the subject's cognitive function comprises an adaptive computer implemented cognitive function test.

22. The method of any of claims 11 to 21, wherein the strategy comprises an elaborative encoding mnemonic.

23. The method of any of claims 11 to 22, wherein the cognitive function test comprises an nback test for assessment of working memory.

24. The method of claim 23, wherein the strategy comprises stages of:
i) memorizing a working set of n steps of the nback;
ii) comparing the current item with the working set;
iii) updating the working set to discard the oldest member.

25. The system of any of claims 1 to 10, configured to perform the method of any one of claims 11 to 24.
